(19) United States
(12) Patent Application Publication
(10) Pub. No.: US 2007/0282394 A1
(43) Pub. Date: Dec. 6, 2007

(54) Assistive listening technology integrated into a Behind-The-Ear sound processor

(76) Inventors: Philip A. Segel, Stevenson Ranch, CA (US); W. Vanbrooks Harrison, Valencia, CA (US); Rankiri Tissa Karunasiri, Castaic, CA (US); Douglas P. Lynch, Valencia, CA (US)

Correspondence Address: ADVANCED BIONICS CORPORATION, 25129 RYE CANYON ROAD, VALENCIA, CA, 91355, US

(21) Appl. No.: 10/938,358

(22) Filed: Sep. 10, 2004

Related U.S. Application Data
(60) Provisional application No. 60/501,978, filed on Sep. 11, 2003.

(51) Int. Cl. A61N 1/00

(52) U.S. Cl. 607/57

(57) ABSTRACT

Various assistive listening devices are coupled to a receiver integrated into a BTE sound processor of a cochlear implant system, or other hearing prosthesis. The assistive listening devices and manner of integration with the BTE sound processor may include, e.g., (a) integration of an FM (frequency modulated) receiver into a remote battery pack; (b) integration of an FM or infrared (IR) or other system into a custom all-in-the-ear hearing instrument shell with an electrical connection through an existing auxiliary connector in the earhook of a BTE sound processor; or (c) integration of an FM system into the BTE sound processor via an interposer module.

The present application claims the benefit of U.S. Provisional Application Ser. No. 60/501,978, filed. 11 Sep. 2003, which application is incorporated herein by reference.

### BACKGROUND OF THE INVENTION

The present application relates to auditory prostheses, and more particularly to techniques, devices and adapters for integrating assisted listening techniques into a Behind-The-Ear (BTE) sound processor, e.g., a BTE sound processor used with a cochlear implant system.

An Assistive Listening Device (ALD) is an electromechanical system designed to improve the signal-to-noise ratio for the listener with a cochlear implant system (or hearing aid). These systems help provide a more complete solution to auditory needs in specific listening situations. Signals from these systems may, with an appropriate electromechanical interface, be routed through a cochlear implant processor, and thereby greatly improve the signal-to-noise ratio for a user of the cochlear implant processor.

An example of an ALD that may assist a user of a cochlear implant processor, or a hearing aid, is a remote microphone that is placed within inches of a speaker and coupled directly to the sound processor of the cochlear implant system, or hearing aid, e.g, through a direct cable wire link. Such ALD thus allows the user of the cochlear implant system, or hearing aid, to readily hear the speaker, despite the presence of other audio signals or background noise that may be present around the user. The speaker may be several feet away from the user, or even several hundred feet (if the direct cable is sufficiently long), yet the user hears the speaker as though the speaker where speaking right next to the user.

In a very broad sense, a telephone is an example of such an ALD. That is, a microphone (the mouth piece of a telephone handset) is placed next to a first person, who is speaking, and who may be very remote from a second person, who is listening. Another example of a simple ALD device, which may be used by a hearing aid user, that uses a direct cable wire link to a remote microphone is the “Pocket Talker”, available commercially from numerous vendors, such as TelTex, Inc. of Kansas City, Mo.

It is known in the art to replace the direct cable wire link, in the above ALD example of using a remote microphone, with a direct frequency modulated (FM) radio frequency (RF) link. With such a system, the output signal from the microphone is sent to an FM transmitter, where it is broadcast as an FM modulated RF signal that can be picked up, or received, by a suitable FM receiver that is carried by the user. The FM receiver, once it picks up and demodulates the broadcast FM signal, sends the received signal to the input circuits of the.cochlear implant system, or other hearing prosthesis. With such a system, a speaker may be located a significant distance form the listener, e.g., at the podium of a large assembly hall, while the user/listener may be located anywhere within range of receiving the FM broadcast signal. See, e.g., U.S. Pat. No. 5,824,022, (audio signals from any source, such as radio. CD-player, tape player or external microphone may be coupled to the cochlear implant via a FM-linked remote control unit 50 through input jack 53), incorporated herein by reference.

Currently available ALDs intended for use with a cochlear implant system, e.g., a BTE sound processor used with a Clarion® CII Bionic Ear cochlear implant system, or a HiReS™ 90K cochlear implant system, manufactured by Advanced Bionics Corporation, of Sylmar, Calif., involve the use of a number of long cables emanating from the BTE sound processor that must be plugged into the ALD. Such cables can be unwieldy, and are also unsightly. Moreover, since the implant manufacturer does not typically manufacture these ALDs, neither the mechanical nor the electrical interface is ideally designed for the end user.

What is needed is an integration of these ALDs into an ear level sound processor, or the development of custom modules that readily interface with a BTE sound processor.

### SUMMARY OF THE INVENTION

The present application addresses the above and other needs by integrating various assistive listening technologies already available commercially into the BTE sound processor of a cochlear implant system, or other hearing prosthesis (e.g, a hearing aid). These BTE sound processors include currently available ear level sound processors, as well as ear level sound processors under development.

The assistive listening devices integrated into a BTE sound processor included within the scope of the present disclosure include, but are not limited to: (a) integration of an FM (frequency modulated) receiver, or other wireless-technology receiver, into a remote battery pack; (b) integration of an FM or infrared (IR) system into a custom all-in-the-ear hearing instrument shell with an electrical connection through an existing auxiliary connector in the earhook of a BTE sound processor; or (c) integration of an FM or other system into the BTE sound processor via an interposer module.

The remote battery pack into which an FM or other receiver may be integrated—one of the options indicated above—may be a body-worn battery pack that carries, e.g., a AAA-sized or AA-sized battery, or a snap-on battery pack that uses a disk battery and that fits over the headpiece, being held in place using a separate magnet.

In accordance with one aspect of the invention, any suitable communication protocol, including those commonly used to facilitate wireless communications between hand-held and other devices, may be used to facilitate reliable and secure communications between the FM receiver that is integrated with the BTE sound processor and a remote FM transmitter.

In accordance with another aspect of the invention, the BTE sound processor into which the assisted listening technologies described herein may be integrated may be a BTE sound processor used as part of a cochlear implant system, a hearing aid system, or other auditory prosthesis.

It is thus a feature of the invention to provide a BTE sound processor into which assisted listening technologies may be easily integrated.

It is a further feature of the invention to allow users of a cochlear implant system to benefit from assisted listening technology.

### BRIEF DESCRIPTION OF THE DRAWINGS

The above and other aspects, features and advantages of the present invention will be more apparent from the following more particular description thereof, presented in conjunction with the following drawings wherein:

FIG. 1 is block diagram of a cochlear implant system in which assisted listening technology is integrated into a BTE system in accordance with the present invention;

FIG. 2 illustrates one manner in which an FM/IR receiver and/or alternative/supplemental power source may be placed or snapped over the headpiece of an existing BTE system in accordance with one embodiment of the invention;

FIG. 3 depicts another manner in which an FM/IR receiver and alternative/supplemental power source may be integrated into a power pack option of a BTE system;

FIG. 4 shows yet another manner in which an FM/IR receiver module may be incorporated into an in-the-ear canal shell which may be integrated into an existing BTE system; and

FIG. 5 shows still another manner in which an ALD interposer module may be interposed into an existing BTE system.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

The present invention will be described in conjunction with a cochlear implant system that uses a Behind-The-Ear (BTE) sound processor. However, it should be emphasized that the invention is not limited to a cochlear implant system that uses a BTE, but may be used with any hearing prosthesis that uses a BTE, or equivalent sound processor, worn on or near the ear of a user.

The following patents and/or patent applications disclose and describe various aspects and features of a cochlear implant system, and are all incorporated herein by reference: U.S. Pat. No. 5,584,869; U.S. Pat. No. 5,824,022; U.S. Pat. No. 6,219,580; U.S. Pat. No. 6,748,094; U.S. Pat. No. 6,775,389; U.S. Patent Application Publication US 2003/0031336 A1, published Feb. 13, 2003; U.S. Patent Application Publication US 2003/0036782 A1, published Feb. 20, 2003; and International Publication WO 97/01314, published Jan. 16, 1997.

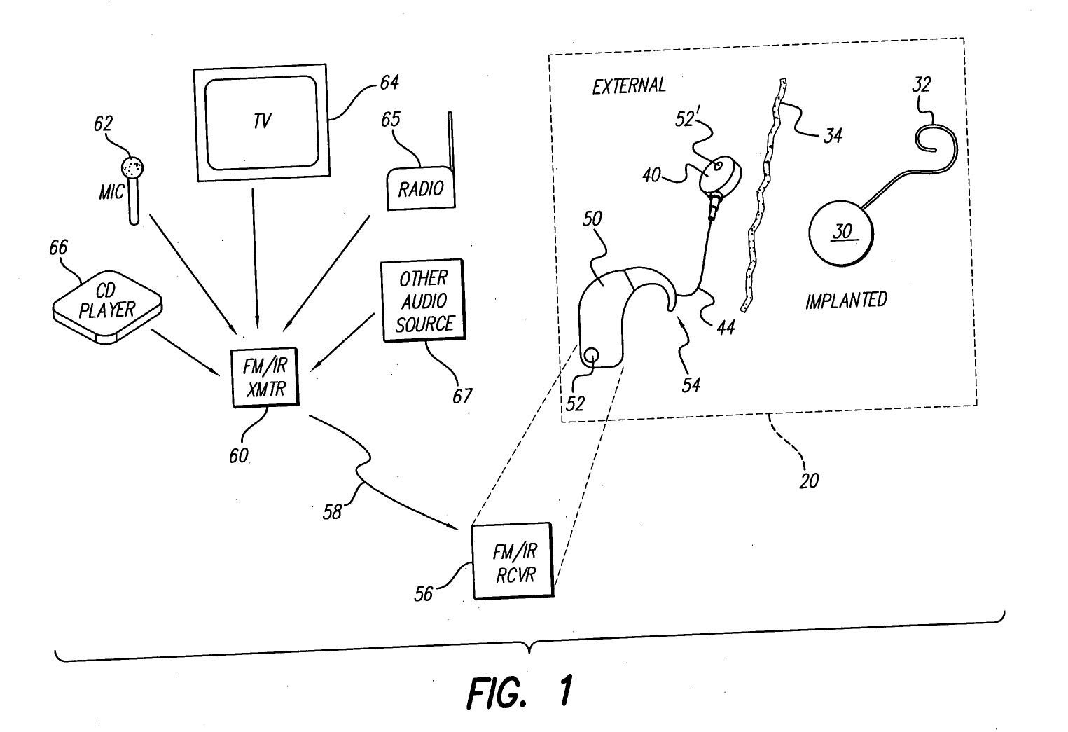

Turning first to FIG. 1, there is shown a block diagram of a cochlear implant system in which assisted listening technology is integrated into a BTE processor used with that system. The cochlear implant system is shown within the dotted line 20 (and hereafter the cochlear implant system will be referred to as the cochlear implant system 20). It should be noted that the various components shown in FIG. 1 are not drawn to scale.

The cochlear implant system 20 includes an implanted portion 30, including an electrode array 32, which are intended to be implanted under the skin 34 of a user of the system. The system 20 further includes an external (non-implantable) headpiece 40 that is connected to a Behind-The-Ear (BTE) processor 50. The BTE processor 50 may include a built-in microphone 52. Alternatively, or conjunctively, the headpiece 40 may include a microphone 52′. Still further, in some embodiments, a supplemental microphone may be detachably connected to the ear hook of the BTE 50, e.g., at location 54, as described in U.S. Patent Application Publication US 2003/0031336 A1, published Feb. 13, 2003.

It should be noted that the BTE processor 50 may, in some embodiments, be part of a housing that is adapted to be worn or carried at a location other than behind the ear. For example, in some embodiments, the processor 50 may be carried in the headpiece 40. In such an embodiment, all of the external components of the cochlear system 20 may be housed within the headpiece 40, or only some of the components, including the speech processor 50, may be housed within the headpiece 40, with other components, such as the battery, being housed elsewhere. In still other embodiments, the processor 50 may be carried in a body-worn housing adapted to be clipped onto the user's clothing, or carried in a pocket contained within the user's clothing. Hence, even though reference has been and will be made throughout this application to the “BTE processor 50”, it is to be understood that the “BTE processor 50” may physically reside in locations other than behind the ear.

The actual electronic circuits that are included within the implantable portion 30 and the external BTE processor portion 50 or headpiece 40 may vary depending upon the particular cochlear implant system that is used. Typically, the headpiece 40 only includes a coil and a magnet, although it may also include a built-in microphone 52′ or, as indicated above, a sound processor. The magnet is used to align the headpiece 40 with a corresponding coil that is included within the implantable portion. See U.S. Pat. No. 6,219,580 for a more complete description of the cochlear implant circuitry and how it is, or may be, partitioned between implantable components and non-implantable components. Such partitioning is not critical to the present invention.

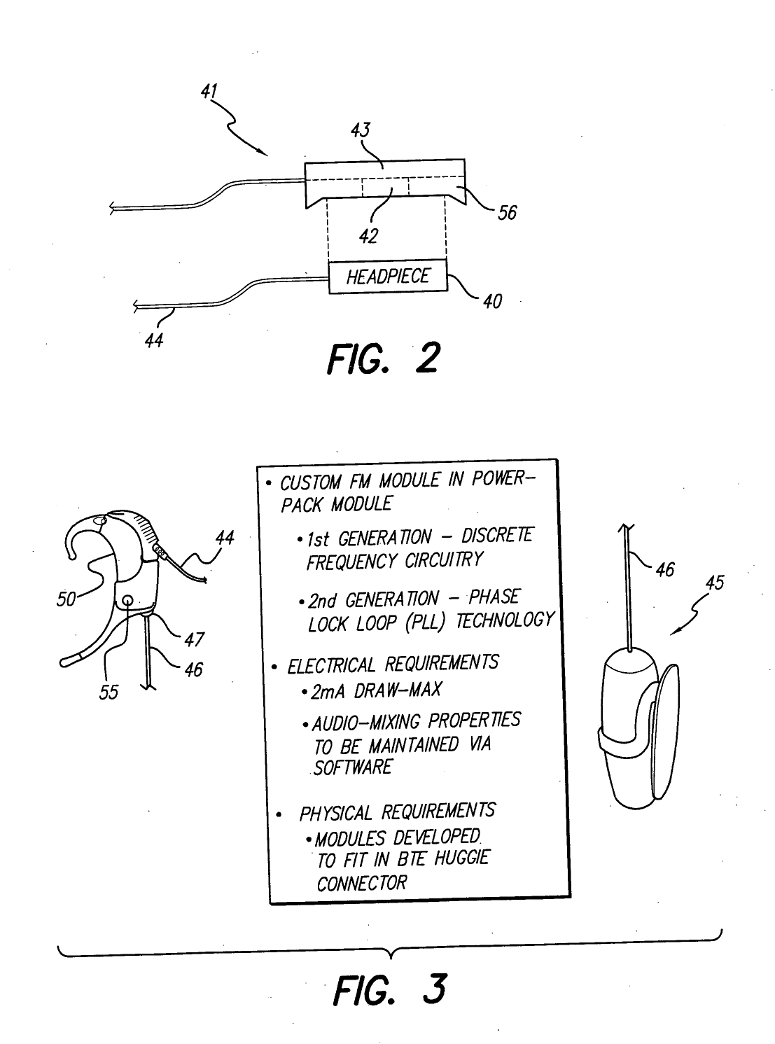

Still with reference to FIG. 1, the present invention integrates an frequency modulated (FM) receiver, or an infrared (IR) receiver, or other type of receiver, shown and identified in FIG. 1 as a FM/IR receiver (RCVR) 56, into the BTE processor 50. Various embodiments of the invention perform the integration of the FM/IR receiver module 56 into the BTE processor 50 in various ways, as described more fully below in conjunction with the description of FIGS. 2-5.

It should be noted that while reference is made herein to an FM or IR receiver, other types of wire-less receivers could also be employed by the invention. For example, any type of radio frequency (RF) receiver could be employed, regardless of the type of modulation used. Further, any type of electromagnetic transmitter may be employed for transmitting appropriately modulated electromagnetic radiation to an electromagnetic receiver, e.g., where electromagnetic radiation (which electromagnetic radiation includes the full spectrum of radiation, from very low frequencies to frequencies that are very high, e.g., beyond visible light) is modulated in an appropriate manner. So long as a modulated carrier signal is transmitted from a remote location, e.g., modulated by information obtained from or through the assistive listening device, and such transmitted carrier signal is reliably received and demodulated by a compatible receiver integrated with the BTE processor, such receiver may be used to help carry out the assistive listening device functions of the present invention.

With a FM/IR receiver module 56 integrated into a BTE processor 50, as shown in FIG. 1, various assisted listening devices (ALDs) or assistive listening technology may be used with the invention. For example, a remote microphone 62 coupled to a suitable remote FM or IR transmitter 60, may be linked to the integrated RM/IR receiver 56 of the BTE processor 50 via communication link 58. Communication link 58 may be a radio frequency (RF) link, or an infrared (IR) link, or other wireless link.

Similarly, a TV 64, a radio 65, a CD player 66, or any other remote audio source 67, may be coupled to the FM/IR transmitter 60, and thereby linked with the BTE processor 50 via the communication link 58 and RM/IR receiver 56. The user of the cochlear implant system 20 may thus be assisted in listening to audio sounds emanating from the remote audio sources.

It is noted that remote audio sources, such as the microphone 62, TV 64, radio 65, CD player 66, or other sources 67, have generally heretofore been coupled to the sound processor 50 by way of a direct cable connection. Such direct cable connection works well, but can become unwieldy and cumbersome, and also limits the distance at which the remote audio source may be located from the user to the length of the cable. In contrast, the communications link 58 provided by the present invention, coupled from the remote audio source directly to the receiver module 56 which is integrated into the BTE processor 50, eliminates the need for such a cable.

From the above description, it is seen that the FM/IR receiver module 56 comprises a key component of the invention. More particularly, the manner in which the FM/IR receiver module 56 is integrated into the BTE processor 50 is an essential part of the invention. In the description that follows, various ways of performing such integration are described. Such ways of integrating the FM/IR receiver module 56 with the BTE processor 50 are given by way of example, and are not intended to be limiting.

Turning to FIG. 2, for example, a supplemental power pack 41 fits over, or snaps onto, the headpiece 40. The headpiece 40 connects with the BTE sound processor 50 by way of cable 44. The power pack 41 may include, in addition to a FM/IR receiver 56, a flat disk battery 43 and a small magnet 42. The small magnet 42 helps secure the pack 41 to its desired location on top of the headpiece 40 because it is attracted to the magnet within the headpiece 40. The disk battery 43 may be readily removed and replaced, as required.

FIG. 3 shows another embodiment of a power pack module 45, known as the PowerPak™ module, which is adapted to be clipped to an article of clothing of the user, in a convenient location. The PowerPak module 45 carries a supplemental battery, e.g., a AAA-sized battery or a AA-sized battery, which is used to power the BTE processor 50 through a cable 46. An FM Receiver is built-into the PowerPak module 45 using discrete frequency circuitry or phase locked loop (PLL) circuitry. Electrical connection with the BTE processor 50 occurs through the cable 46. The cable 46 mates with a connector 47, termed the BTE “huggie” connector, that already forms part of the BTE processor 50. (One such connector that may be used with a BTE processor is described more fully in U.S. Pat. No. 6,748,094, previously incorporated herein by reference.)

The BTE processor 50 may include a visual status indicator light 55 that indicates when an FM signal is being received through the cable 46, i.e., through the FM receiver included within the PowerPak module 45. Such visual indication is helpful to an audiologist or other personnel in the vicinity of the user as confirmation that an FM link has been established from a remote source. The use of a similar indicator is described in U.S. Pat. No. 5,584,869.

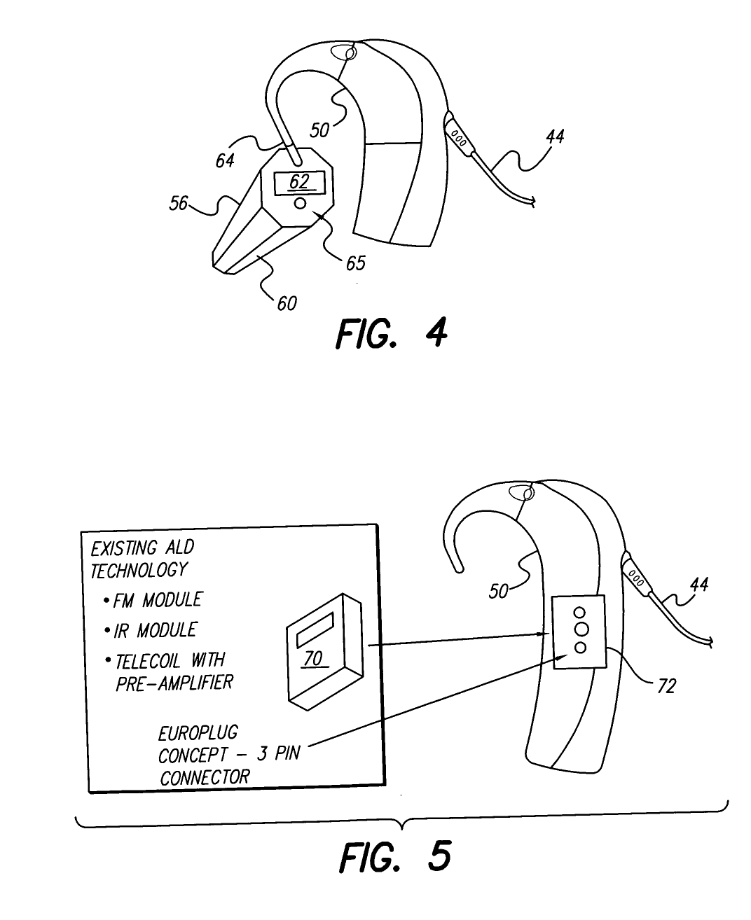

FIG. 4 shows yet another manner in which an FM/IR receiver module 56 may be incorporated into an in-the-canal (ITC) shell housing 60. In addition to the FM/IR receiver 56, an additional power source, e.g., a battery 62, may also be incorporated within the ITC shell 60. The circuits and battery within the shell 60 electrically connect with a stalk member 64 adapted to detachably fit onto the ear-hook side of the BTE processor 50. A gain control button, or knob, or receiver port (for gain control signals that are generated using an IR remote control) may also be incorporated into the circuits housed within the shell 60, as shown generally at location 65.

FIG. 5 shows still another manner in which an ALD interposer plug module 72 may be interposed into an existing BTE processor 50. The module 72 may incorporate a 3 pin connector, e.g., the same or similar to that which is known as the Europlug connector, so as to allow existing ALD devices 70 to be detachably plugged into the BTE processor 50. The ALD devices 70 may include, e.g., a FM module, an IR module, or a Telecoil with pre-amplifier.

The interposer module 72 mates with the connector used by the BTE processor 50 to connect a battery module thereto. An exemplary interposer module, although showing a slightly different type of connector, is described in U.S. Patent Application Publication US 2003/0036782 A1.

As described above, it is thus seen that the present invention provides a BTE sound processor into which assisted listening technologies may be easily integrated. It is also seen that the invention allows users of a cochlear implant system, or other auditory prosthesis, to more readily benefit from existing, or yet to be developed, assisted listening technology.

While the invention herein disclosed has been described by means of specific embodiments and applications thereof, numerous modifications and variations could be made thereto by those skilled in the art without departing from the scope of the invention set forth in the claims.

1-15. (canceled)

16. In a cochlear implant system that includes a body-worn sound processor and an implanted portion, an improved system for interfacing with assistive listening devices, comprising:
an electrode array connected to the implanted portion;
an external headpiece coupled to the body-worn sound processor, said external headpiece including means for aligning the external headpiece with the implanted portion;
an electromagnetic receiver integrated into the sound processor;
an electromagnetic transmitter at a location remote from the electromagnetic receiver, said electromagnetic transmitter including means for transmitting electromagnetic radiation modulated with audio information obtained from an assistive listening device (ALD);
wherein the electromagnetic receiver includes
means for receiving the transmitted electromagnetic radiation,
means for demodulating the electromagnetic radiation in order to recover the audio information therefrom,
means for presenting the audio information as an input signal; and
means for transmitting the processed input signal from the sound processor to the implanted portion wherein the electrode array connected to the implanted portion applies the processed signal as an electrical stimulation signal to a patient's cochlea.

17. The cochlear implant system of claim 16 wherein the body-worn sound processor comprises a behind-the-ear (BTE) sound processor, and further wherein the electromagnetic receiver comprises a radio frequency (RF) receiver that may be detachably secured to the external headpiece.

18. The cochlear implant system of claim 16 wherein the body-worn sound processor has a battery pack module coupled thereto, and further wherein the electromagnetic receiver comprises a radio frequency (RF) receiver that may be detachably secured to the battery pack module.

19. The cochlear implant system of claim 16 wherein the body-worn sound processor has an interposer module attached thereto, and further wherein the electromagnetic receiver comprises a radio frequency (RF) receiver located within the interposer module.

20. The cochlear implant system of claim 16 wherein the electromagnetic radiation transmitted by the electromagnetic transducer comprises radio frequency (RF) or infrared (IR) radiation.

21. The cochlear implant system of claim 18 wherein the battery pack module further includes a magnet, and wherein the battery pack module is held in place over the headpiece by magnetic forces.

22. The cochlear implant system of claim 18 wherein the battery pack module further includes a flat disk battery.

23. The cochlear implant system of claim 19 wherein the interposer module includes a connector module that interfaces with the ALD.

24. The cochlear implant system of claim 23 wherein the connector module comprises a Europlug 3-pin connector.

25. The cochlear implant system of claim 23 wherein the ALD that interfaces with the connector module is selected from the group comprising an FM module, an IR module, and a telecoil with pre-amplifier module.

26. In a cochlear implant system that includes a body-worn sound processor and an implanted portion, an improved system for interfacing with assistive listening devices, comprising:
an electrode array connected to the implanted portion;
an external headpiece coupled to the body-worn sound processor, said external headpiece including means for aligning the external headpiece with the implanted portion;
an electromagnetic receiver integrated into the sound processor;
an electromagnetic transmitter at a location remote from the electromagnetic receiver, said electromagnetic transmitter including means for transmitting electromagnetic radiation modulated with audio information obtained from an assistive listening device (ALD), wherein the ALD includes a telecoil with pre-amplifier module;
wherein the electromagnetic receiver includes
means for receiving the transmitted electromagnetic radiation,
means for demodulating the electromagnetic radiation in order to recover the audio information therefrom,
means for presenting the audio information as an input signal to the cochlear; and
means for transmitting the processed input signal from the sound processor to the implanted portion, wherein the electrode array connected to the implanted portion applies the processed signal as an electrical stimulation signal to a patient's cochlea.
